(19) United States
(12) Patent Application Publication
(10) Pub. No.: US 2008/0160598 A1
(43) Pub. Date: Jul. 3, 2008

(54) Organic monolith reactor and the preparation method thereof

(76) Inventors: Osamu Nozaki, Osakasayama-shi (JP); Hiroko Kawamoto, Yonago-shi (JP); Motonori Munesue, Matsubara-shi (JP)

Correspondence Address: FLYNN THIEL BOUTELL & TANIS, P.C., 2026 RAMBLING ROAD, KALAMAZOO, MI, 49008-1631, US

(21) Appl. No.: 11/881,428

(22) Filed: Jul. 27, 2007

(30) Foreign Application Priority Data
Dec. 28, 2006 (JP) JP2006-356320

(51) Int. Cl. C12M 1/00

(52) U.S. Cl. 435/283.1

(57) ABSTRACT

The present invention provides an organic monolith reactor and the preparation method thereof, wherein the reactor is possible to manufacture even into a small size and even at room temperature, and the manufacturing process is simple and relatively short period of time is required.

In an organic monolith reactor according to the present invention, a horseradish peroxidase is embedded onto a microscopic area with a photopolymerized polymer. The method of preparing an organic monolith reactor according to the present invention comprises (a) dispersing a horseradish peroxidase in a mixed solution of a photopolymerizable monomer and a photopolymerizing agent, (b) introducing the dispersed solution into a microscopic area, and (c) performing a photo-irradiation onto the microscopic area at room temperature.

### TECHNICAL FIELD

The present invention relates to an organic monolith reactor that may be used for the measurement of various ingredients such as hydrogen peroxide, glucose and cholesterol in human body fluids, and the preparation method thereof.

### BACKGROUND OF THE INVENTION

Examples of the methods for measuring hydrogen peroxide include flow injection-horseradish peroxidase (FI-HRP), catalyst chemiluminescence method (Chemiluminescence; CL), and etc.

In the conventional CL method, two ends of a reactor tube have been immobilized with frit by packing a HRP immobilized gel in the reactor tube.

For example, they are immobilized to a HRP immobilized stationary phase by using amino group-introduced gel such as pearl beads, glass beads, chitosan gel, polystyrene gel, acryl gel as the HRP immobilized stationary phase, and diluting HRP in a buffer solution of for example phosphoric acid, according to Nakane's method (a method for oxidizing a sugar chain). Moreover, an adopted method is to pack the HRP immobilized stationary phase in a fluorinated resin tube and to fix two ends of the tube with frit made of an appropriate material (patent reference 1).

That is, for the manufacture of a reactor, this method requires three steps of (1) immobilizing HRP to a gel, (2) packing a column of a HRP immobilized gel, and (3) frit-closing of column (patent reference 1; Publication of Japanese patent application No. 2004-81138).

However, the conventional CL method has a problem that a small-sized reactor is difficult to manufacture. According to the conventional CL method, a reactor tube is packed after HRP is immobilized to an immobilized stationary phase of beads or gel. It makes packing is difficult or even impossible especially when the diameter of the reactor tube is relatively small.

The conventional CL method has further problems that it requires three steps for manufacturing of the reactor and some steps may not be performed at room temperature. A longer period needed for the manufacture of a reactor is also a problem of the conventional CL method.

### SUMMARY OF THE INVENTION

Therefore, in order to overcome the aforementioned conventional problems, the present invention aims to provide an organic monolith reactor and the preparation method thereof, wherein the reactor is possible to manufacture even into a small size and even at the room temperature, and the manufacturing process is simple and relatively short period of time is required.

In an organic monolith reactor according to the present invention, a horseradish peroxidase is embedded in a microscopic area with a photopolymerized polymer.

Further, in an organic monolith reactor herein, a horseradish peroxidase and a biological material are embedded in a microscopic area with a photopolymerized polymer.

A method of preparing an organic monolith reactor according to the present invention comprises (a) dispersing a horseradish peroxidase in a mixed solution of a photopolymerizable monomer and a photopolymerizing agent, (b) introducing the dispersed solution into a microscopic area, and (c) performing a photo-irradiation onto the microscopic area at the room temperature.

Further, a method of preparing an organic monolith reactor according to the present invention comprises (a) dispersing a horseradish peroxidase and a biological material in a mixed solution of a photopolymerizable monomer and a photopolymerizing agent, (b) introducing the dispersed solution into a microscopic area, and (c) performing a photo-irradiation onto the microscopic area at the room temperature.

In the present invention, at least one selected from the group consisting of an enzyme such as glucose oxidase, cholesterol oxidase, alcohol oxidase, L-amino acid oxidase, uricase and monoamine oxidase; a protein; and a DNA probe may be used as the biological material.

In the present invention, any of the group consisting of a fluorinated resin tube, a capillary glass tube, a hematocrit capillary tube, a microstructure prepared on a silicon wafer or a glass plate, an ultrafine glass tube, a waterdrop-shaped monolith lump formed on a flat plate, micropores of a gel surface, an inner wall of a micro titer well, and an inner wall of a test tube may be used as a microscopic area.

Due to the aforementioned construction, a reactor according to the present invention is possible to manufacture even into a small size and even at room temperature, and the manufacturing process is simple and relatively short period of time is required.

### DETAILED DESCRIPTION OF THE INVENTION

Hereunder is provided a detailed description of embodiments according to the present invention.

In an organic monolith reactor according to the present invention, a horseradish peroxidase (referred to as “HRP” hereinafter) is embedded in a microscopic area with a photopolymerized polymer.

Further, in an organic monolith reactor herein, a horseradish peroxidase and a biological material are embedded in a microscopic area with a photopolymerized polymer.

Further, in an organic monolith reactor, HRP and a biological material are embedded onto a microscopic area with a photopolymerized polymer in such a manner that the central part in the microscopic area may be hollow.

In the present invention, a plastic tube (e.g. a fluorinated resin tube), a capillary glass tube, a hematocrit capillary tube, a microstructure prepared on a silicon wafer or a glass plate, an ultrafine glass tube, a waterdrop-shaped monolith lump formed on a flat plate, micropores of a gel surface, an inner wall of a micro titer well, and an inner wall of a test tube may be used as a microscopic area. The microscopic area may be so determined that (i) a diameter (when a fluorinated resin tube, a glass tube or a hematocrit capillary tube is used), (ii) a width (when a groove is used), or (iii) a diameter (when a hole is used) is several tens of micrometers (μm), respectively.

A method of preparing an organic monolith reactor according to the present invention comprises (a) dispersing HRP and a biological material in a mixed solution containing a photopolymerizable monomer or an oligomer or a mixture thereof and a photopolymerizing agent, (b) introducing the dispersed solution into a microscopic area, and (c) performing a photo-irradiation onto the microscopic area.

Further, a method of preparing an organic monolith reactor according to the present invention comprises (a) dispersing HRP and a biological material in a mixed solution of a photopolymerizable monomer and a photopolymerizing agent, (b) introducing the dispersed solution into a microscopic area, and (c) performing a photo-irradiation onto the microscopic area.

In the present invention, a radical polymerizable monomer may be used as a photopolymerizable monomer, and examples of the radical polymerizable monomer include monofunctional acrylate and multifunctional acrylate. Further, the use of it also includes an oligomer. Examples of an oligomer include an epoxy acrylate, an urethane acrylate, a polyester acrylate, a polyether acrylate, a polybutadiene acrylate, a copolymeric acrylate and a silicone acrylate.

In the present invention, an acetophenone-based compound, a benzoin ether based compound, a benzyl ketal based compound or a ketone based compound may be used as a photopolymerizing agent.

In the present invention, at least one selected from the group consisting of an enzyme such as glucose oxidase, cholesterol oxidase, alcohol oxidase, amino acid oxidase, uricase and monoamine oxidase; a protein; and a DNA probe may be used as a biological material.

For example, when a glucose oxidase is used as the biological material, the glucose is oxidized to generate a gluconolactone and a hydrogen peroxide. Thus, the quantification of the hydrogen peroxide by HRP makes the measurement of the glucose content in human body fluids possible. Because a cholesterol oxidase decomposes a cholesterol to produce a hydrogen peroxide, the quantification of the hydrogen peroxide by HRP makes possible the measurement of the cholesterol content in human body fluids. Further, because an alcohol oxidase decomposes an alcohol to produce a hydrogen peroxide, the quantification of the hydrogen peroxide by HRP makes possible the measurement of the alcohol content in human body fluids. Furthermore, because an amino acid oxidase produces a hydrogen peroxide while separating ammonia from an amino acid, the quantification of the hydrogen peroxide by HRP makes possible the measurement of the amino acid content in human body fluids.

In the present invention, when a microscopic area is a tube such as a capillary glass tube and a hematocrit capillary tube, HRP and/or a biological material are embedded onto a wall of the tube by the photo-irradiation, thus rendering the central part hollow and changing the back pressure of the reactor into nearly zero.

Next, in the method according to the present invention, the optimum mixing ratio of HRP to the biological material was calculated as described below when HRP and a biological material are dispersed in a mixed solution of a photopolymerizable monomer and a photopolymerizing agent.

First, the resolution in quantifying glucose was investigated as a function of a glucose oxidase (referred to as “GOD” hereinafter) content (3, 6, 12, 18, 24 mg) while maintaining the HRP amount into a constant value (14 mg) when GOD was used as a biological material.

As a result, it is possible to observe how the reactivity to a glucose sample changes as the ratio of GOD to HRP in the reactor increases. Because the reactivity was ascertained to be good when 12 mg of GOD was used relative to 14 mg of HRP, the same weight of HRP and a biological material (weight ratio=1:1) was selected as the optimum mixing ratio.

### EXAMPLES

The present invention is described more specifically by the following Examples. Examples herein are meant only to illustrate the present invention, but in no way to limit the claimed invention.

### Example 1

### Preparation of Organic Monolith Reactor

HRP was dispersed in a mixed solution of 3-methacryloxypropyl trimethoxy silane (a photopolymerizable monomer) and 2-hydroxy-2-methyl-1-phenyl propan-1-one (a photoinitiator; DAROCUR1173™, Ciba Specialty Chemicals Corp.) as an acetophenone-based compound, and was introduced into a hematocrit capillary tube (a microscopic area; Terumo Co. Ltd.; length 75 mm, outer diameter 1.5 mm). Photo-irradiation was performed onto this hematocrit capillary tube at room temperature for 12 hours by using a UV irradiator (Chemco Corp.). The tube was cut into the length of 18 mm, thus providing an organic monolith reactor embedded with HRP (referred to as “HRP reactor” hereinafter).

### Test Example 1

### Measurement of Hydrogen Peroxide

The HRP reactor prepared in Example 1 was equipped onto a cell holder of a flow-type chemiluminometer or chemiluminescence device (JASCO Corp.). After washed with a mobile phase solution for about one hour, this was used for measurement of hydrogen peroxide.

A hydrogen peroxide sample (25 μL) was injected to a mobile phase flow S1 (BSA 0.01% solution, 100 μL/min) with an auto sampler. Another mobile phase S2 (imidazole tricine solution, pH 9.4, 100 μL/min) was added to the mobile phase flow S1, and injected into a flow cell reactor, thus causing chemiluminescence in the flow cell reactor.

The chemiluminescence may be detected by a photo-multiplayer of the chemiluminometer. Hence, hydrogen peroxide in human body fluids may be quantified when a calibration curve is prepared after obtaining chemiluminescence values at various concentrations of hydrogen peroxide.

### Example 2

### Preparation of Organic Monolith Reactor

In each of a mixed solution of 3-methacryloxypropyl trimethoxy silane (a photopolymerizable monomer) and diethoxy acetophenone (a photoinitiator; IRGACURE1800™, Ciba Specialty Chemicals Corp.) as an acetophenone-based compound were dispersed a mixture of glucose oxidase (GOD) and HRP (weight ratio=1:1). The dispersed solution was introduced onto the capillary glass tube (a microscopic area; length 75 mm, inner diameter 0.85 mm), and photo-irradiation was performed onto the capillary tube at room temperature for 12 hours by using a UV irradiator (Chemco Corp.). The tube was cut into the length of 18 mm, thus providing an organic monolith reactor embedded with GOD and HRP (referred to as “GOD/HRP reactor” hereinafter).

### Example 3

### Preparation of Organic Monolith Reactor

In each of a mixed solution of 3-methacryloxypropyl trimethoxy silane (a photopolymerizable monomer) and diethoxy acetophenone (a photoinitiator; IRGACURE1800™, Ciba Specialty Chemicals Corp.) as an acetophenone-based compound were dispersed a mixture of cholesterol oxidase (COD) and HRP (weight ratio=1:1). The dispersed solution was introduced onto the capillary glass tube (a microscopic area; length 75 mm, inner diameter 0.85 mm), and photo-irradiation was performed onto the capillary tube at room temperature for 12 hours by using a UV irradiator (Chemco Corp.). The tube was cut into the length of 18 mm, thus providing an organic monolith reactor embedded with COD and HRP (referred to as “COD/HRP reactor” hereinafter).

### Example 4

### Preparation of Organic Monolith Reactor

In each of a mixed solution of 3-methacryloxypropyl trimethoxy silane (a photopolymerizable monomer) and diethoxy acetophenone (a photoinitiator; IRGACURE1800™, Ciba Specialty Chemicals Corp.) as an acetophenone-based compound were dispersed a mixture of alcohol oxidase (ALO) and HRP (weight ratio=1:1). The dispersed solution was introduced onto the capillary glass tube (a microscopic area; length 75 mm, inner diameter 0.85 mm), and photo-irradiation was performed onto the capillary tube at room temperature for 12 hours by using a UV irradiator (Chemco Corp.). The tube was cut into the length of 18 mm, thus providing an organic monolith reactor embedded with ALO and HRP (referred to as “ALO/HRP reactor” hereinafter).

### Example 5

### Preparation of Organic Monolith Reactor

In each of a mixed solution of 3-methacryloxypropyl trimethoxy silane (a photopolymerizable monomer) and diethoxy acetophenone (a photoinitiator; IRGACURE1800™, Ciba Specialty Chemicals Corp.) as an acetophenone-based compound were dispersed a mixture of amino acid oxidase (AMO) and HRP (weight ratio=1:1). The dispersed solution was introduced onto the capillary glass tube (a microscopic area; length 75 mm, inner diameter 0.85 mm), and photo-irradiation was performed onto the capillary tube at room temperature for 12 hours by using a UV irradiator (Chemco Corp.). The tube was cut into the length of 18 mm, thus providing an organic monolith reactor embedded with AMO and HRP (referred to as “AMO/HRP reactor” hereinafter).

### Test Example 2

### Measurement of Glucose

The GOD/HRP reactor prepared in Example 2 was equipped onto a cell holder of a flow-type chemiluminometer (JASCO Corp.). After washed with a mobile phase solution for about one hour, this was used for measurement of glucose.

A glucose sample (50 mM/L) was injected to a mobile phase flow S1 (BSA 0.05% solution, 100 μL/min) with an auto sampler. Another mobile phase S2 (imidazole tricine solution, pH 8.6, 100 μL/min) was added to the mobile phase flow S1, and injected into a flow cell reactor, thus causing chemiluminescence in the flow cell reactor.

The chemiluminescence may be detected by a photo-multiplayer of the chemiluminometer. Hence, glucose in human body fluids may be quantified when a calibration curve is prepared after obtaining chemiluminescence values at various concentrations of glucose.

### Test Example 3

### Measurement of Cholesterol

The COD/HRP reactor prepared in Example 3 was equipped onto a shell holder of a flow-type chemiluminometer (JASCO Corp.). After washed with a mobile phase solution for about one hour, this was used for measurement of cholesterol.

A cholesterol sample (3.5 mg/mL) was injected to a mobile phase flow S1 (BSA 0.05% solution, 100 μL/min) with an auto sampler. Another mobile phase S2 (imidazole tricine solution, pH 8.6, 100 μL/min) was added to the mobile phase flow S1, and injected into a flow cell reactor, thus causing chemiluminescence in the flow cell reactor.

The chemiluminescence may be detected by a photo-multiplayer of the chemiluminometer. Hence, cholesterol in human body fluids may be quantified when a calibration curve is prepared after obtaining chemiluminescence values at various concentrations of cholesterol.

### Test Example 4

### Measurement of Alcohol

The ALO/HRP reactor prepared in Example 4 was equipped onto a shell holder of a flow-type chemiluminometer (JASCO Corp.). After washed with a mobile phase solution for about one hour, this was used for measurement of alcohol.

An ethyl alcohol sample (25%) was injected to a mobile phase flow S1 (BSA 0.05% solution, 100 μL/min) with an auto sampler. Another mobile phase S2 (imidazole tricine solution, pH 8.6, 100 μL/min) was added to the mobile phase flow S1, and injected into a flow cell reactor, thus causing chemiluminescence in the flow cell reactor.

The chemiluminescence may be detected by a photo-multiplayer of the chemiluminometer. Hence, alcohol in human body fluids may be quantified when a calibration curve is prepared after obtaining chemiluminescence values at various concentrations of ethyl alcohol.

### Test Example 5

### Measurement of Amino Acid

The AMO/HRP reactor prepared in Example 5 was equipped onto a shell holder of a flow-type chemiluminometer (JASCO Corp.). After washed with a mobile phase solution for about one hour, this was used for measurement of amino acid.

A leucine sample (2.5 mg/mL) was injected to a mobile phase flow S1 (BSA 0.05% solution, 100 μL/min) with an auto sampler. Another mobile phase S2 (imidazole tricine solution, pH 8.6, 100 μL/min) was added to the mobile phase flow S1, and injected into a flow cell reactor, thus causing chemiluminescence in the flow cell reactor.

The chemiluminescence may be detected by a photo-multiplayer of the chemiluminometer. Hence, amino acid in human body fluids may be quantified when a calibration curve is prepared after obtaining chemiluminescence values at various concentrations of leucine.

1. An organic monolith reactor, wherein a horseradish peroxidase is embedded in a microscopic area with a photopolymerized polymer.

2. The organic monolith reactor, wherein a horseradish peroxidase and a biological material are embedded in a microscopic area with a photopolymerized polymer.

3. A method for preparing an organic monolith reactor, which comprises:
(a) dispersing a horseradish peroxidase in a mixed solution of a photopolymerizable monomer and a photopolymerizing agent,
(b) introducing the dispersed solution into a microscopic area, and
(c) performing a photo-irradiation onto the microscopic area at room temperature.

4. A method for preparing an organic monolith reactor, which comprises:
(a) dispersing a horseradish peroxidase and a biological material in a mixed solution of a photopolymerizable monomer and a photopolymerizing agent,
(b) introducing the dispersed solution into a microscopic area, and
(c) performing a photo-irradiation onto the microscopic area at room temperature.

5. The organic monolith reactor of claim 2, wherein the biological material is at least one selected from the group consisting of an enzyme selected from the group consisting of glucose oxidase, cholesterol oxidase, alcohol oxidase, L-amino acid oxidase, uricase and monoamine oxidase; a protein; and a DNA probe.

6. The method of claim 4, wherein the biological material is at least one selected from the group consisting of an enzyme selected from the group consisting of glucose oxidase, cholesterol oxidase, alcohol oxidase, L-amino acid oxidase, uricase and monoamine oxidase; a protein; and a DNA probe.

7. The organic monolith reactor of claim 1, wherein the microscopic area is selected from the group consisting of a fluorinated resin tube, a capillary glass tube, a hematocrit capillary tube, a microstructure prepared on a silicon wafer or a glass plate, an ultrafine glass tube, a waterdrop-shaped monolith lump formed on a flat plate, micropores of a gel surface, an inner wall of a micro titer well, and an inner wall of a test tube.

8. The method of claim 3, wherein the microscopic area is selected from the group consisting of a fluorinated resin tube, a capillary glass tube, a hematocrit capillary tube, a microstructure prepared on a silicon wafer or a glass plate, an ultrafine glass tube, a waterdrop-shaped monolith lump formed on a flat plate, micropores of a gel surface, an inner wall of a micro titer well, and an inner wall of a test tube.
